(19) United States
(12) Patent Application Publication
(10) Pub. No.: US 2026/0308137 A1
(43) Pub. Date: Oct. 8, 2026

(54) A METHOD AND PLANT FOR PURIFYING ETHYLENE CARBONATE

(71) Applicant: SULZER MANAGEMENT AG, Winterthur (CH)

(72) Inventors: Etienne RIGAUT, Riedisheim (FR); Cecilia MONDELLI, Zürich (CH); Erik TEMMEL, Basel (CH); Yongle LI, Shanghai (CN); Severine DETTE, Winterthur (CH)

(21) Appl. No.: 19/484,726

(22) PCT Filed: May 24, 2024
(86) PCT No.: PCT/EP2024/064361

§ 371 Date: Nov. 14, 2025

(30) Foreign Application Priority Data
May 26, 2023 (WO) PCT/CN2023/096542
Jun. 9, 2023 (EP) 23178428.1

(51) Int. Cl. C07D 317/38; B01D 3/14; B01D 9/00

(52) U.S. Cl. CPC C07D 317/38; B01D 3/143; B01D 9/0059

(57) ABSTRACT

A method for purifying ethylene carbonate from a crude ethylene carbonate composition includes subjecting a crude composition containing ethylene carbonate to a distillation step so as to obtain a pre-purified composition containing at least 99.5% by weight of ethylene carbonate and subjecting the pre-purified composition to a melt crystallization step so as to obtain a purified ethylene carbonate composition. The method is performed in a plant comprising a distillation column, a vessel, a crystallizer and a feed line for a crude composition containing ethylene carbonate. The distillation column includes an overhead outlet line, a bottom outlet line and a side outlet line. The crystallizer includes an inlet line for a pre-purified composition and an outlet line for purified ethylene carbonate composition. The side outlet line of the distillation column is connected with the inlet line for pre-purified composition of the at least one crystallizer.

### CROSS REFERENCE TO RELATED APPLICATION

This application is a U.S. national stage application of International Application No. PCT/EP2024/064361, filed on May 24, 2024 which claims priority to European Patent Application No. 23178428.1 which was filed on Jun. 9, 2023 and International application No. PCT/CN2023/096542 which was filed on May 26, 2023, the contents of each of which are hereby incorporated by reference.

### TECHNICAL FIELD

The present disclosure relates to a method and a plant for purifying ethylene carbonate.

### BACKGROUND

Ethylene carbonate is an important industrial raw material, which is used for instance as solvent or as starting material for the synthesis of ethylene glycols, dimethyl carbonate, ethylene glycol ethers and ethanolamines. Ethylene glycol, in turn, is widely used as chemical, such as for example as antifreeze agent or as monomer in the production of polyester and polyethylene terephthalate, as liquid coolant and as solvent, whereas dimethyl carbonate is used for the manufacture of polycarbonates. Furthermore, ethylene carbonate is gaining more and more importance as electrolyte solvent for lithium-ion batteries, which facilitates the transport of lithium ions from the cathode to the anode. Thereby, ethylene carbonate contributes in a two-fold manner to combat climate change. Firstly, the major route applied in the industrial synthesis of ethylene carbonate is the reaction of ethylene oxide with carbon dioxide, which allows consumption of captured carbon dioxide as a feedstock for green chemicals production, and secondly lithium-ion batteries contribute to the decarbonization of the energy sector through electrification, especially in relation to transport.

### SUMMARY

It has been determined however, considering that high contents of glycols and, especially, of water are detrimental for the long-term performance of batteries, since they promote the formation of hydrogen fluoride, which in turn affects the quality of the solid electrode interface, ethylene carbonate used as electrolyte solvent for lithium-ion batteries needs to be very pure. Common techniques for purifying crude ethylene carbonate are distillation, crystallization, absorption or the like. However, these methods have at least one of the drawbacks of a low separation efficiency such as but not limited to low depletion factors for glycols and/or water, of a high energy demand and of subjecting the ethylene carbonate to some thermal stress leading to its accelerated degradation and formation of by-products and undesired impurities.

In view thereof, the object underlying the present disclosure is to provide a method and a plant for purifying ethylene carbonate being characterized by a particular high productivity, which is the product mass purified per unit of time, by a high recovery yield and by a particular low energy consumption, wherein the method further leads to very pure ethylene carbonate containing 50 ppm or less impurities, such as water and glycols.

In accordance with the present disclosure, this object is satisfied by providing a method for purifying ethylene carbonate from a crude ethylene carbonate composition comprising the steps of:
a) subjecting a crude composition containing ethylene carbonate to at least one distillation step so as to obtain a pre-purified composition containing at least 99.5% by weight of ethylene carbonate and
b) subjecting the pre-purified composition obtained in step a) to at least one melt crystallization step so as to obtain a purified ethylene carbonate composition, wherein the method is performed in a plant comprising at least one distillation column, one vessel, at least one crystallizer and a feed line for a crude composition containing ethylene carbonate, wherein the distillation column, if the plant comprises one distillation column, or the first distillation column, if the plant comprises two or more distillation columns, comprises an overhead outlet line, a bottom outlet line and a side outlet line, wherein the at least one crystallizer comprises an inlet line for a pre-purified composition and an outlet line for purified ethylene carbonate composition, wherein the side outlet line of the distillation column is directly or indirectly connected with the inlet line for pre-purified composition of the at least one crystallizer, wherein
   i) either the vessel is connected with the feed line for a crude composition containing ethylene carbonate and with an inlet line, wherein the inlet line is connected with a gas outlet of a first reboiler, wherein the first reboiler is further connected with the bottom outlet line of the distillation column, wherein the vessel further comprises an outlet line leading via a second reboiler to an inlet line of the distillation column, wherein the bottom fraction of the distillation column being withdrawn from the distillation column via the bottom outlet line is partially evaporated in the first reboiler so as to obtain a gaseous fraction and a liquid fraction, or
   ii) the feed line for a crude composition containing ethylene carbonate leads into the distillation column, wherein the bottom outlet line of the distillation column leads via a first reboiler to the vessel, wherein the vessel comprises a gas outlet being connected with an inlet line of the distillation column and the vessel comprises a liquid outlet being connected with a line leading into a second reboiler, wherein the second reboiler comprises an outlet for gas being connected with an inlet line of the distillation column, wherein the bottom fraction of the distillation column being withdrawn from the distillation column via the bottom outlet line is partially evaporated in the first reboiler, wherein at least a portion of the gaseous fraction obtained in the first reboiler is recycled into the distillation column via the inlet line.

This solution bases on the finding that by first subjecting a crude ethylene carbonate containing composition, which can for instance originate from a reaction route or can be recovered from any kind of recycling stream, to one distillation step being performed in a distillation column or to two or more distillation steps being performed in two or more distillation columns, wherein the distillation step(s) lead(s) to a pre-purified composition containing at least 99.5% by weight of ethylene carbonate, before the pre-purified composition is subjected to one or more melt crystallization steps, not only very pure ethylene carbonate containing at most 50 ppm impurities or even not more than 10 ppm impurities, such as water and glycols, is obtained, but also the method can be performed with a particular low energy consumption, nevertheless with a particular high productivity and with a high recovery yield. This is among others due to the fact that the distillation step(s) can be performed under comparable mild conditions, since the crude ethylene carbonate composition is only purified therein to an ethylene carbonate content of at least 99.5% by weight. This does not only allow to keep the ethylene carbonate recovery rate at more than 90%, but also to maintain the energy consumption at a low level, because more stringent distillation conditions or further distillation steps consuming much more energy for achieving a higher purity degree of ethylene carbonate, such as ethylene carbonate containing 200 to 800 ppm impurities, are not necessary. Such a high purity can be achieved by distillation only with an energy consumption being between 3 and 6 times higher compared to the energy consumption of a distillation step being performed under mild conditions so as to obtain a pre-purified composition containing at least 99.5% by weight of ethylene carbonate. In addition, by leading the bottom fraction of the distillation column through a reboiler and by partially evaporating the bottom fraction therein, before at least a portion of the gaseous fraction or of the liquid fraction obtained therein is led to another reboiler and evaporated therein, before at least a portion of the gaseous fraction obtained in the second reboiler is recycled into the distillation column, the energy consumption of the process is significantly reduced even when maintaining the particular high productivity and a high recovery yield. This is due to the fact that thereby the bottom fraction being withdrawn via the bottom outlet line is separated into a fraction having a higher content of high boiling compounds (meaning compounds having a higher boiling point than ethylene carbonate, such a low or medium molecular weight polymer, catalyst ionic liquid and salt, e.g. calcium and sodium salts) and a lower content of ethylene carbonate than the other fraction, wherein the fraction having a higher content of high boiling compounds and a lower content of ethylene carbonate is withdrawn form the process, whereas the other fraction having a lower content of high boiling compounds and a higher content of ethylene carbonate is directly or indirectly recycled into the distillation column. On account thereof, a back-mixing of both fractions is avoided, which in turn allows to increase the separation efficiency within the distillation column and to reduce the energy consumption of the process. In addition, thereby an efficient removal of especially the high boiling compounds from the crude ethylene carbonate composition is achieved, which leads to a reduction of the degradation of ethylene carbonate in the pre-purified composition in the subsequent step(s) and thus to an increase of the ethylene carbonate yield of about 20% during the process. The further purification of the pre-purified composition is then performed in accordance with the present disclosure in one or more melt crystallization steps, which leads to purified ethylene carbonate containing at most 50 ppm or even at most 10 ppm impurities. It has been found in the present disclosure that melt crystallization is characterized by a high separation efficiency for ethylene carbonate from water and glycols for each crystallization stage as well as by very high depletion factors of significantly higher than 10 for glycols, if a pre-purified composition containing at least 99.5% by weight of ethylene carbonate is used as starting composition for the melt crystallization. Depletion factor means with this regard the ratio of the concentration of a substance in the feed of a melt crystallization stage divided by the concentration of the product as obtained as the result of the same melt crystallization stage. Even if it would be possible to purify the crude ethylene carbonate composition solely by melt crystallization without any distillation step, this would require a very high number of crystallization stages in order to obtain a purification to an impurity content of at most 50 ppm. Such a high number of crystallization stages would lead to very high operational costs due to a particular high energy consumption. Furthermore, this would require a large size of the crystallization apparatus. Thus, the distillation step(s) a) leading to just a pre-purified composition containing at least 99.5% by weight of ethylene carbonate and the crystallization step(s) b) synergistically work together and lead with a particular high productivity and with a high recovery yield to a very pure ethylene carbonate containing at most 50 ppm impurities or even not more than 10 ppm impurities at a particular low energy consumption. More specifically, the energy savings are 20 to 25% compared to the known methods.

That the side outlet line of the distillation column is connected with the inlet line for a pre-purified composition of the at least one crystallizer means a direct or indirect connection of the side outlet line of the distillation column with the inlet line for a pre-purified composition of the at least one crystallizer. An indirect connection means in particular that the side outlet line of the distillation column leads as feed line into a second distillation column comprising an overhead outlet line, a side outlet line and a bottom outlet line, wherein preferably the side outlet line is directly connected with the inlet line for a pre-purified composition of the at least one crystallizer.

As set out above, step a) is intentionally performed so as to obtain a pre-purified composition containing at least 99.5% by weight of ethylene carbonate, i.e. a composition, which needs to be not so pure to have only low ppm contents of impurities. Thereby, the number of distillation steps can be kept low as well as the temperature and pressure conditions during the distillation step(s) can be kept moderate so that the energy demand for the distillation is quite low. Good results are in particular obtained, when in step a) at least one distillation step is performed so as to obtain a pre-purified composition containing 99.5 to 99.9% by weight of ethylene carbonate. The content of glycol impurities in the pre-purified composition is preferably 100 to 2,000 ppm. More preferably, in step a) at least one distillation step is performed so as to obtain a pre-purified composition containing 99.6 to 99.9% by weight, even more preferably of 99.7 to 99.9% by weight, still more preferably of 99.8 to 99.9% by weight and most preferably of 99.85 to 99.90% by weight of ethylene carbonate.

In a further development of the idea of the present disclosure, it is suggested that the crude composition used in step a) contains 10 to 99% by weight, preferably 50 to 99% by weight, more preferably 70 to 99% by weight and most preferably 95 to 99% by weight of ethylene carbonate.

As set out above, step a) comprises at least one distillation step. Thus, step a) can comprise two or more distillation steps. However, it is preferred that step a) does not comprise more than two distillation steps. For instance, step a) comprises two subsequent distillation steps. The crude composition is preferably fed in this embodiment in step a) into a first distillation column and is distilled therein into an overhead fraction, a side fraction and a bottom fraction, wherein the bottom fraction of the first distillation column is led into a second distillation column and is distilled therein into an overhead fraction, a side fraction and a bottom fraction, wherein the overhead fraction of the second distillation column is led as the pre-purified composition to the at least one melt crystallization step of step b). However, it is more preferred in this embodiment that the crude composition is fed in step a) into a first distillation column and is distilled therein into an overhead fraction, into a bottom fraction and into a side fraction, wherein the side fraction of the first distillation column is led into a second distillation column and is distilled therein into an overhead fraction, into a bottom fraction and into a side fraction, wherein the side fraction of the second distillation column is led as the pre-purified composition to the at least one melt crystallization step of step b).

In accordance with an alternative particularly preferred embodiment of the present disclosure, step a) comprises (only) one distillation step being performed in a distillation column being connected with a vessel and two reboilers as described above, wherein the crude composition is fed in step a) into the distillation column and is distilled therein into an overhead fraction, into a bottom fraction and preferably also into a side fraction, wherein preferably the side fraction is led as the pre-purified composition to the at least one melt crystallization step of step b).

According to option i) of the present disclosure, the vessel is connected with the feed line for a crude composition containing ethylene carbonate and with an inlet line, wherein the inlet line is connected with a gas outlet of a first reboiler, wherein the first reboiler is further connected with the bottom outlet line of the distillation column, wherein the vessel further comprises an outlet line leading via a second reboiler to an inlet line of the distillation column. The bottom fraction of the distillation column being withdrawn from the distillation column via the bottom outlet line is partially evaporated in the first reboiler so as to obtain a gaseous fraction and a liquid fraction, wherein at least a portion of the gaseous fraction is led via the inlet line being connected with a gas outlet of the first reboiler into the vessel. It is further preferred in this embodiment that the first reboiler comprises a liquid outlet line, through which the liquid fraction obtained in the first reboiler is withdrawn from the process. In addition, the first reboiler can comprise a further gas outlet, through which a fraction containing low boiling compounds is withdrawn from the process. Good results are in particular obtained in this embodiment, when the first reboiler is a wiped film evaporator, a falling film evaporator or a short path evaporator, more preferably when the first reboiler is a wiped film evaporator or a short path evaporator and most preferably when the first reboiler is a wiped film evaporator. In addition, the first reboiler can comprise two or more heat exchangers in series. The second reboiler is preferably a falling film evaporator.

In a further development of the idea of the present disclosure, it is suggested that the vessel is further connected in this embodiment with an outlet line of the distillation column, wherein the distillation column further comprises one or more beds of structured packings, wherein below the lowermost bed of structured packings a collector is arranged, which has a collection line being connected with the outlet line. Instead of the one or more beds of structured packings or in addition to the one or more beds of structured packings, the distillation column can comprise one or more beds of random packings and/or one or more trays.

According to option ii) of the present disclosure, the feed line for a crude composition containing ethylene carbonate leads into the distillation column, wherein the bottom outlet line of the distillation column leads via a first reboiler to the vessel, wherein the vessel comprises a gas outlet being connected with an inlet line of the distillation column and the vessel comprises a liquid outlet being connected with a line leading into a second reboiler, wherein the second reboiler comprises an outlet for gas being connected with an inlet line of the distillation column, wherein the bottom fraction of the distillation column being withdrawn from the distillation column via the bottom outlet line is partially evaporated in the first reboiler, wherein at least a portion of the gaseous fraction obtained in the first reboiler is recycled into the distillation column via the inlet line. It is further preferred in this embodiment that the liquid fraction obtained in the vessel is partially evaporated in the second reboiler, wherein at least a portion of the gaseous fraction obtained in the second reboiler is recycled into the distillation column via the outlet for gas of the second reboiler and via the inlet line of the distillation column being connected therewith. In addition, it is preferred in this embodiment that the second reboiler further comprises a liquid outlet line for high boiling compounds and a gas outlet line for low boiling compounds. Good results are in particular obtained in this embodiment, when the first reboiler is a falling film evaporator, whereas it is preferred that the second reboiler is a wiped film evaporator, a falling film evaporator or a short path evaporator, more preferably when the second reboiler is a wiped film evaporator or a short path evaporator and most preferably when the second reboiler is a wiped film evaporator. In addition, the second reboiler can comprise two or more heat exchangers in series.

In a further development of the idea of the present disclosure, it is preferred that the overhead of the distillation column comprises an outlet line and an inlet line, both being connected with a recycle line leading from the outlet line to the inlet line, wherein the recycle line is connected with an overhead condenser or cold trap, respectively, and downstream thereof with a gas-liquid separator, wherein the gas-liquid separator is connected with a liquid line being connected with the recycle line and wherein the gas-liquid separator is connected with a gas line.

Good results are in particular obtained, when the sidewall of the distillation column comprises an outlet line and an inlet line, both being connected with a recycle line leading from the outlet line to the inlet line, wherein the recycle line is connected with a side condenser and downstream thereof with a gas-liquid separator, wherein the gas-liquid separator is connected with a liquid line being connected with the recycle line and wherein the gas-liquid separator is connected with a gas line, wherein the liquid line leads back into the distillation column.

In order to minimize the energy consumption during the method, it is further proposed to perform the distillation step(s) so as to allow heat integration. For instance, the distillation is performed in the at least one distillation step so that the condensation temperature of the overhead fraction is at least 100° C., preferably 110 to 150° C. and more preferably 115 to 125° C. This allows generation of in the overhead condenser a heat transfer medium stream with a temperature of at least 80° C., preferably of 90 to 130° C. and more preferably of 95 to 105° C. This heat transfer medium can in turn be used to power another step, such as the sweating and/or melting of the ethylene carbonate crystals obtained in the at least one melt crystallization step, as described further below. This allows a reduction in the overall energy consumption by up to 25%, compared to a respective system without implementation of any heat integration.

Furthermore, it is preferred that the distillation column(s) used in step a) comprise(s) one or more internals, such as one or more beds of structured packings, one or more beds of random packings or one or more trays. This leads to a particular efficient mass and heat transfer between the descending liquid phase and the ascending gas phase. More preferably, the distillation column(s) used in step a) comprise(s) one or more beds of structured packings, such as one to five and preferably two to four beds of structured packings. Good results are in particular obtained, when each of the structured packings has a specific surface area 100 to 750 m²/m³ and more preferably of 150 to 500 m²/m³.

The present disclosure is neither particularly limited concerning the kind of at least one melt crystallization stage nor concerning the number of melt crystallization stages. Good results are in particular obtained, when the at least one melt crystallization step b) comprises at least one melt crystallization stage being selected from the group consisting of falling film crystallization stages, static crystallization stages or suspension crystallization stages. Falling film crystallization means also dynamic crystallization. Each of the aforementioned crystallization techniques can comprise one to five, preferably one to four, more preferably one to three and most preferably one to two melt crystallization stages.

In accordance with a particularly preferred embodiment of the present disclosure, the at least one melt crystallization step b) comprises at least one falling film crystallization stage. During a falling film crystallization stage, melt to be crystallized flows along a cooled surface downwards, such as along the inside of a cooled tube, which allows crystals to grow from the falling film of melt on the inside surface of the tube, which is cooled by a falling film of a cooling agent co-currently flowing on the outside surface of the tube. High and very reproducible transfer rates are achieved on both side of the tube, wherein the resulting shear at the crystal/liquid interface transports impurities rapidly into the bulk of the melt. Preferably, the falling film crystallizer used for the crystallization stage(s) b) contains a plurality of vertical tubes, in which the crystal layers grow as cylindrical shells, a collection vessel beneath the tubes as well as a circulating pump. Before the start of a crystallization stage, the collection vessel is filled with a batch of the melt to be crystallized. The circulating pump is then started to wet the tubes and initiate crystallization at a certain temperature level, while coolant temperature ramping is started. The melt circulation rate is adjusted to a high value compared to the rate of crystal deposition so that conditions of temperature and composition are approximately uniform across the length of the tubes. The temperature is ramped down at a constant rate until the collecting vessel level drops to a preset value. At this point, melt circulation is discontinued.

Good results are in particular obtained, when step b) comprises one to five, preferably one to four, more preferably one to three and most preferably one to two falling film crystallization stages.

Preferably, the pre-purified composition obtained in step a) and subjected in step b) to at least one melt crystallization step is fed into a first of the two to five falling film crystallization stages so as to produce a first ethylene carbonate enriched crystallized fraction and a residue fraction, wherein the first ethylene carbonate enriched crystallized fraction is fed into a second of the two to five falling film crystallization stages, wherein in any of the second and of the optional third to five falling film crystallization stages an ethylene carbonate enriched crystallized fraction and a residue fraction is produced, wherein each of the ethylene carbonate enriched crystallized fractions produced in the second and the optional third to fourth falling film crystallization stages is fed into a downstream ethylene carbonate crystallization stage and each of the residue fractions produced in the second and the optional third to fifth falling film crystallization stages is fed into an upstream falling film crystallization stage, wherein the ethylene carbonate enriched crystallized fraction obtained in the most downstream of the falling film crystallization stages is the purified ethylene carbonate composition.

In addition, it is preferred that the production of an ethylene carbonate enriched crystallized fraction and of a residue fraction in a crystallization stage comprises the steps of removing the remaining liquid from the crystallization stage as residue fraction after termination of the crystallization in the crystallization stage, of melting the crystal layer obtained in the crystallization stage and of withdrawing the obtained crystal melt as ethylene carbonate enriched crystallized fraction from the crystallization stage.

In a further development of the idea of the present disclosure, it is suggested that before melting the crystal layer obtained in the crystallization stage, one or more sweating steps of the crystal layer are carried out so as to obtain one or more sweating fractions and a purified crystal layer, wherein preferably at least a portion of the first sweating fraction obtained thereby is fed to the remaining liquid which has been removed as residue fraction. Sweating is achieved by increasing the temperature of the crystals to a numeric value being just below the melting point of ethylene carbonate, such as 0.1 to 2° C. below the melting point of ethylene carbonate, in order to liquefy impurities and assist further draining.

Good results are in particular obtained, when at least one and preferably all of the melt crystallization step(s) is/are performed at a temperature of −10° C. to 70° C., preferably at a temperature of −5° C. to 65° C. and more preferably at a temperature of 0° C. to 60° C.

In accordance with a particular preferred embodiment of the present disclosure, the purified ethylene carbonate composition comprises 50 ppm or less and preferably 10 ppm or less impurities.

In a further aspect, the present disclosure relates to a plant for purifying ethylene carbonate from a crude ethylene carbonate composition, wherein the plant comprises at least one distillation column, one vessel, at least one crystallizer and a feed line for a crude composition containing ethylene carbonate, wherein the distillation column, if the plant comprises one distillation column, or the first distillation column, if the plant comprises two or more distillation columns, comprises an overhead outlet line, a bottom outlet line and a side outlet line, wherein the at least one crystallizer comprises an inlet line for a pre-purified composition and an outlet line for purified ethylene carbonate composition, wherein the side outlet line of the first distillation column is directly or indirectly connected with the inlet line for pre-purified composition of the at least one crystallizer, wherein
i) either the vessel is connected with the feed line for a crude composition containing ethylene carbonate and with an inlet line, wherein the inlet line is connected with a gas outlet of a first reboiler, wherein the first reboiler is further connected with the bottom outlet line of the distillation column, wherein the vessel further comprises an outlet line leading via a second reboiler to an inlet line of the distillation column, or
ii) the feed line for a crude composition containing ethylene carbonate leads into the distillation column, wherein the bottom outlet line of the distillation column leads via a first reboiler to the vessel, wherein the vessel comprises a gas outlet being connected with an inlet line of the distillation column and the vessel comprises a liquid outlet being connected with a line leading into a second reboiler, wherein the second reboiler comprises an outlet for gas being connected with an inlet line of the distillation column.

Good results are in particular obtained for option i), when the vessel is further connected with an outlet line of the distillation column, wherein the second reboiler further comprises a liquid outlet line for high boiling compounds and a gas outlet line for low boiling compounds.

In a further development of the idea of the present disclosure, it is proposed for option i) that the distillation column further comprises one or more beds of structured packings, wherein below the lowermost bed of structured packings a collector is arranged, which has a collection line being connected with the outlet line. Instead of the one or more beds of structured packings or in addition to the one or more beds of structured packings, the distillation column can comprise one or more beds of random packings and/or one or more trays.

In accordance with a further preferred embodiment of the present disclosure, it is preferred that in the embodiment of option ii) the second reboiler further comprises a liquid outlet line for high boiling compounds and a gas outlet line for low boiling compounds.

Preferably, the plant comprises two distillation columns, wherein the second distillation column comprises an overhead outlet line, a bottom outlet line and a side outlet line, wherein the side outlet line of the second distillation column is directly connected with the inlet line for pre-purified composition of the at least one crystallizer, and wherein the side outlet of the first distillation column is connected with an inlet of the second distillation column by a connection line.

Good results are in particular obtained, when the at least one crystallizer comprises one to five, preferably one to four, more preferably one to three and most preferably one to two falling film crystallization stages.

### BRIEF DESCRIPTION OF THE DRAWINGS

The disclosure will be explained in more detail hereinafter with reference to the drawings, in which:

FIG. 1 schematically shows a plant for purifying ethylene carbonate from a crude ethylene carbonate composition in accordance with one embodiment of the present disclosure.

FIG. 2 schematically shows a plant for purifying ethylene carbonate from a crude ethylene carbonate composition in accordance with another embodiment of the present disclosure.

FIG. 3 schematically shows a plant for purifying ethylene carbonate from a crude ethylene carbonate composition in accordance with another embodiment of the present disclosure.

### DETAILED DESCRIPTION

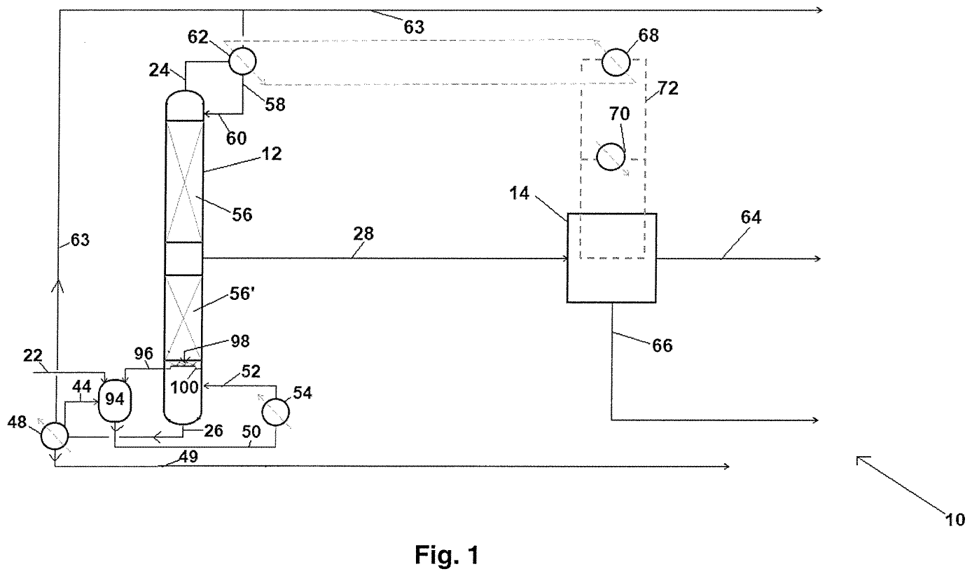

The plant 10 for purifying ethylene carbonate from a crude ethylene carbonate composition shown in FIG. 1 comprises a distillation column 12, a vessel 94 and a crystallizer 14. More specifically, the plant 10 comprises an inlet line 22 for a crude composition containing ethylene carbonate and the distillation column 12 comprises an overhead outlet line 24, a bottom outlet line 26 and a side outlet line 28 for pre-purified composition to be led into the crystallizer 14. Furthermore, the crystallizer 14 comprises an inlet line for a pre-purified composition and an outlet line 64 for purified ethylene carbonate composition, wherein the side outlet line 28 of the distillation column 12 is directly connected with the inlet line for pre-purified composition of the crystallizer 14. In turn, the vessel 94 is connected with the feed line 22 for the crude composition containing ethylene carbonate and with an inlet line 44, wherein the inlet line 44 is connected with a gas outlet of a first reboiler 48. The first reboiler 48 is further connected with the bottom outlet line 26 of the distillation column 12, wherein the vessel 94 further comprises an outlet line 50, which leads via a second reboiler 54 to an inlet line 52 of the distillation column 12. In addition, the vessel 94 is further connected with an outlet line 96 of the distillation column 12 and the first reboiler 48 further comprises a liquid outlet line 49 for high boiling compounds and a gas outlet line 63 for low boiling compounds. In fact, the liquid outlet line 49 of the first reboiler 48 for high boiling compounds is a withdrawal line leading out of the plant 10. Moreover, the distillation column 12 comprises two beds of structured packing 56, 56′, one 56 of which being arranged above the side outlet line 28 and the other 56′ of which being arranged below the side outlet line 28. Below the lower bed of structured packing 56′ a collector 98 is provided, which has a collection line 100, which is connected with the outlet line 96 of the distillation column 12. Via the collection line 100, a portion of the liquid descending through the lower bed of structured packing 56′ flows into the outlet line 96, whereas the remaining portion thereof further descends into the bottom section of the distillation column 12 and from there into the bottom outlet line 26. In addition, the outlet line 24 of the overhead of the distillation column 12 is connected with a recycle line 58, which in turn is connected with an inlet line 60 leading into the top portion of the distillation column 12. In turn, the recycle line 58 is connected with an overhead condenser 62, which is in addition connected with the outlet line 63 for lights.

The crystallizer 14 comprises three falling film crystallization stages, an outlet line 64 for pure ethylene carbonate and an outlet line 66 for residue. Furthermore, the crystallizer 14 is connected with a heater 68 and a cooler 70, which form together with the condenser 62 a heat integration system 72.

During the operation, a crude composition containing ethylene carbonate is fed as feed via inlet line 22 into the vessel 94, in which it is mixed with the liquid fraction having been collected by the collector 98, from which a portion is withdrawn via the outlet line 96 and led therefrom into the vessel 94. The remaining portion of the liquid fraction further descends into the bottom section of the distillation column 12 and flows from there into the bottom outlet line 26, from which it is led into the first reboiler 48. In the first reboiler 48, the liquid fraction is partially evaporated so that a vapor fraction with reduced content of high boiling compounds is obtained and a portion thereof is led via line 44 into the vessel 94 and another portion thereof is withdrawn from the process via the outlet line 63 for low boiling compounds. Furthermore, a liquid fraction with an increased content of high boiling compounds is obtained in the first reboiler 48, which is withdrawn from the process via the liquid outlet line for high boiling compounds 49. The mixture obtained in the vessel 94 is fed via line 50 into the second reboiler 54, in which it is evaporated and then led via the inlet line 52 into the bottom section of the distillation column 12. The vapor generated during the distillation is withdrawn from the distillation column via the overhead outlet line 24, which is then partially condensed in the overhead condenser 62, from which the liquid portion is refluxed via recycle line 58 into the distillation column 12, whereas the vapor portion obtained in the overhead condenser 62 is withdrawn from the process via the outlet line 63 for low boiling compounds. Furthermore, a side fraction is withdrawn from the distillation column 12 via the side outlet line for pre-purified composition having an ethylene carbonate content of 99.5 to 99.9% by weight, which is fed into the crystallizer 14, in which the pre-purified composition is further purified. While the purified ethylene carbonate is withdrawn from the process via the outlet line 64 for pure ethylene carbonate, the residue obtained during the crystallization is withdrawn from the process via the outlet line 68 for residue.

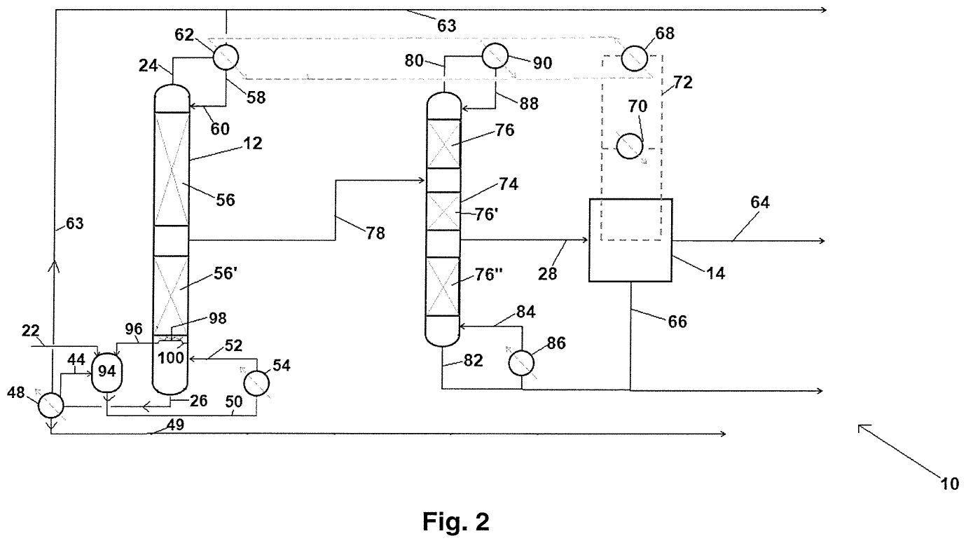

The plant shown in FIG. 2 correspond to that of FIG. 1 except that it further comprises a second distillation column 74, which is arranged between the first distillation column 12 and the crystallizer 14. More specifically, the second distillation column 74 comprises three beds of structured packings 76, 76′, 76″, wherein a connection line 78 leads from the side outlet of the first distillation column 12 to the second distillation column 74 and enters the second distillation column 74 at a location between the uppermost and middle beds of structured packings 76, 76′. Also the second distillation column 74 comprises an overhead outlet 80, a bottom outlet 82 and further comprises a recycle line 84 being provided with a reboiler 86 and leading from the bottom outlet line 82 back into the bottom portion of the second distillation column 74. Likewise thereto, the second distillation column 74 further comprises a recycle line 88 being provided with a condenser 90 and leading from the overhead outlet line 80 back into the overhead portion of the second distillation column 74.

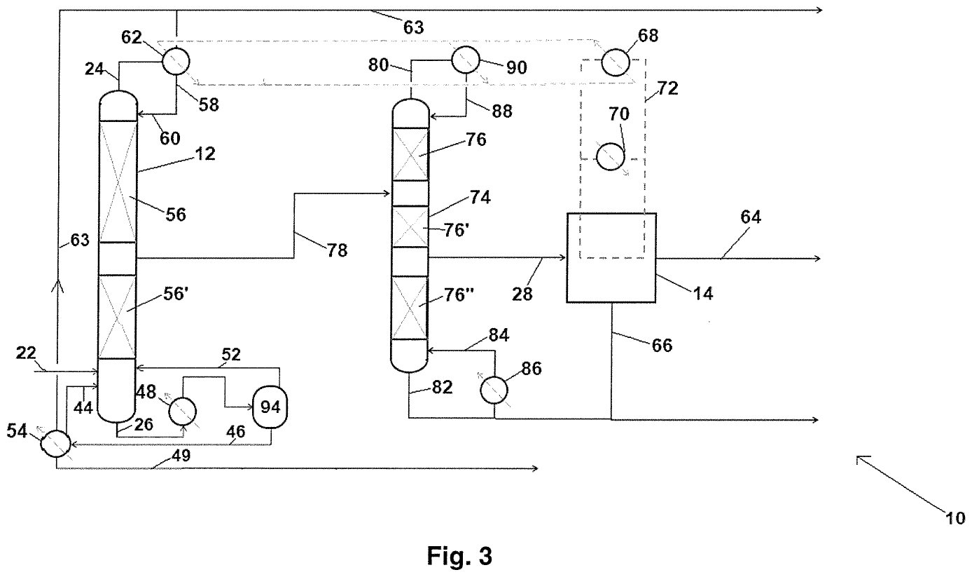

FIG. 3 shows an alternative embodiment of the plant according to option ii). In the embodiment shown in FIG. 3, the inlet line 22 is not—as in the embodiment shown in FIG. 2—connected with the vessel 94, but is directly connected with the first distillation column 12. Moreover, the first distillation column 12 does not—as in the embodiment shown in FIG. 2—comprise a collector 98 and no collection line 100. Rather, the bottom outlet line 26 of the distillation column 12 leads via a first reboiler 48 to the vessel 94, wherein the vessel 94 comprises a gas outlet being connected with an inlet line 52 of the distillation column 12 and the vessel 94 further comprises a liquid outlet being connected with a line 46 leading into a second reboiler 54. The second reboiler 54 comprises an outlet for gas being connected with an inlet line 44 of the distillation column 12, a further outlet for gas being connected with a gas outlet line 63 for low boiling compounds and a liquid outlet line 49 for high boiling compounds.

1. A method for purifying ethylene carbonate from a crude ethylene carbonate composition comprising:
subjecting a crude composition containing ethylene carbonate to at least one distillation step so as to obtain a pre-purified composition containing at least 99.5% by weight of ethylene carbonate;
subjecting the pre-purified composition to at least one melt crystallization step so as to obtain a purified ethylene carbonate composition; and
performing the method in a plant comprising at least one distillation column, one vessel, at least one crystallizer and a feed line for the crude composition containing ethylene carbonate, the at least one distillation column, if when the plant comprises one distillation column, or the first distillation column, if when the plant comprises two or more distillation columns, comprises an overhead outlet line, a bottom outlet line and a side outlet line, the at least one crystallizer comprises an inlet line for the pre-purified composition and an outlet line for the purified ethylene carbonate composition, the side outlet line of the at least one distillation column is directly or indirectly connected with the inlet line for the pre-purified composition of the at least one crystallizer,
either the vessel is connected with the feed line for the crude composition containing ethylene carbonate and with an inlet line, the inlet line is connected with a gas outlet of a first reboiler, the first reboiler is further connected with the bottom outlet line of the distillation column, the vessel further comprises an outlet line leading via a second reboiler to an inlet line of the at least one distillation column, a bottom fraction of the at least one distillation column being withdrawn from the at least one distillation column via the bottom outlet line is partially evaporated in the first reboiler so as to obtain a gaseous fraction and a liquid fraction, or
the feed line for the crude composition containing ethylene carbonate leads into the at least one distillation column, the bottom outlet line of the at least one distillation column leads via the first reboiler to the vessel, wherein the vessel comprises a gas outlet connected with the inlet line of the at least one distillation column and the vessel comprises a liquid outlet connected with a line leading into the second reboiler, the second reboiler comprises an outlet for gas connected with an the inlet line of the at least one distillation column, the bottom fraction of the at least one distillation column being withdrawn from the at least one distillation column via the bottom outlet line is partially evaporated in the first reboiler, and at least a portion of the gaseous fraction obtained in the first reboiler is recycled into the at least one distillation column via the inlet line

2. The method in accordance with claim 1, wherein when the plant comprises two or more distillation columns, the crude composition is fed into the first distillation column and is distilled therein into an overhead fraction, into the bottom fraction and into a side fraction, the side fraction of the first distillation column is led into a second distillation column and is distilled therein into an overhead fraction, into a bottom fraction and into a side fraction, the side fraction of the second distillation column is led as the pre-purified composition to the at least one crystallizer so as to perform therein the at least one melt crystallization step-of

3. The method in accordance with claim 1, or wherein when the vessel is connected with the feed line for the crude composition containing ethylene carbonate, the vessel is further connected with an outlet line of the distillation column, wherein-the first reboiler further comprises a liquid outlet line for high boiling compounds and a gas outlet line for low boiling compounds, the at least one distillation column further comprises one or more beds of structured packings, and below a lowermost bed of the structured packings a collector is arranged, the collector having a collection line being connected with the outlet line of the distillation column.

4. The method in accordance with claim, wherein when the plant comprises two or more distillation columns, an overhead of the first distillation column comprises the overhead outlet line and an inlet line both the overhead outlet line and the inlet line being connected with a recycle line leading from the overhead outlet line to the inlet line, the recycle line is connected with an overhead condenser and downstream thereof with a gas-liquid separator, the gas-liquid separator is connected with a liquid line being connected with the recycle line and the gas-liquid separator is connected with a gas line.

5. The method in accordance with claim 1, wherein when the plant comprises two or more distillation columns, a sidewall of the first distillation column comprises an outlet line and an inlet line, both the outlet line and the inlet line of the sidewall being connected with a recycle line leading from the outlet line to the inlet line, wherein the recycle line is connected with a side condenser and downstream thereof with a gas-liquid separator, the gas-liquid separator is connected with a liquid line and is connected with a gas line, wherein-and the liquid line leads into the first distillation column.

6. The method in accordance with claim 1, wherein the at least one melt crystallization step comprises at least one melt crystallization stage being selected from the group consisting of falling film crystallization stage, static crystallization stage and suspension crystallization stage, and comprises at least one falling film crystallization stage.

7. The method in accordance with claim 1, wherein step b)the subjecting the pre-purified composition to at least one melt crystallization step comprises one to five falling film crystallization stages.

8. The method in accordance with claim 7, wherein the pre-purified composition and subjected to at least one melt crystallization step is fed into a first of the two to five falling film crystallization stages so as to produce a first ethylene carbonate enriched crystallized fraction and a residue fraction, the first ethylene carbonate enriched crystallized fraction is fed into a second of the two to five falling film crystallization stages, in any of the second and third to fifth falling film crystallization stages further ethylene carbonate enriched crystallized fractions and further residue fractions are produced, each of the further ethylene carbonate enriched crystallized fractions produced in the second and the third to fourth falling film crystallization stages is fed into a downstream ethylene carbonate crystallization stage and each of the further residue fractions produced in the second and the third to five falling film crystallization stages is fed into an upstream falling film crystallization stage, the ethylene carbonate enriched crystallized fraction of the a most downstream of the falling film crystallization stages is the purified ethylene carbonate composition.

9. The method in accordance with claim 8, wherein producing the ethylene carbonate enriched crystallized fraction and the residue fraction in a crystallization stage comprises removing a remaining liquid from the crystallization stage as the residue fraction after termination of crystallization in the crystallization stage, of melting the crystal layer obtained in the crystallization stage and of withdrawing the obtained crystal melt as ethylene carbonate enriched crystallized fraction from the crystallization stage, wherein before melting the crystal layer obtained in the crystallization stage, one or more sweating steps of the crystal layer are carried out so as to obtain one or more sweating fractions and a purified crystal layer.

10. The method in accordance with claim 1, wherein the purified ethylene carbonate composition comprises 50 ppm or less.

11. A plant for purifying ethylene carbonate from a crude ethylene carbonate composition, the plant comprising:
at least one distillation column;
one vessel;
at least one crystallizer; and
a feed line for a crude composition containing ethylene carbonate, the at least one distillation column, when the plant comprises one distillation column or the first distillation column, when the plant comprises two or more distillation columns, comprises an overhead outlet line, a bottom outlet line and a side outlet line the at least one crystallizer comprises an inlet line for a pre-purified composition and an outlet line for purified ethylene carbonate composition, the side outlet line of the first distillation column is directly or indirectly connected with the inlet line for pre-purified composition of the at least one crystallizer,
either the vessel is connected with the feed line for the crude composition containing ethylene carbonate and with an inlet line, the inlet line is connected with a gas outlet of a first reboiler, first reboiler is further connected with the bottom outlet line of the at least one distillation column, the vessel further comprises an outlet line leading via a second reboiler to an inlet line of the at least one distillation column, or
the feed line for the crude composition containing ethylene carbonate leads into the at least one distillation column, the bottom outlet line of the at least one distillation column leads via the first reboiler to the vessel, wherein the vessel comprises a gas outlet connected with an the inlet line of the at least one distillation column and the vessel comprises a liquid outlet being connected with a line leading into the second reboiler, and the second reboiler comprises an outlet for gas connected with the inlet line of the distillation column.

12. The plant in accordance with claim 11, wherein when the vessel is connected with the feed line for the crude composition containing ethylene carbonate, the vessel is further connected with an outlet line of the at least one distillation column, and the first reboiler further comprises a liquid outlet line for high boiling compounds and a gas outlet line for low boiling compounds.

13. The plant in accordance with claim 11, wherein the at least one distillation column further comprises one or more beds of structured packings, below a lowermost bed of structured packings collector is arranged, the collector having a collection line connected with the outlet line.

14. The plant in accordance with claim 11, wherein ii)when the feed line for the crude composition containing ethylene carbonate leads into the at least one distillation column, the second reboiler further comprises a liquid outlet line for high boiling compounds and a gas outlet line for low boiling compounds.

15. The plant in accordance with claim 11, wherein when the at least one distillation column includes two distillation columns, the second distillation column comprises an overhead outlet line, a bottom outlet line and a side outlet line, the side outlet line of the second distillation column is directly connected with the inlet line for pre-purified composition of the at least one crystallizer, and the side outlet of the first distillation column is connected with an inlet of the second distillation column by a connection line.

16. The method in accordance with claim 1, wherein the subjecting the pre-purified composition to at least one melt crystallization step comprises one to two falling film crystallization stages.

17. The method in accordance with claim 1, wherein the purified ethylene carbonate composition comprises 10 ppm or less.
